(19) United States
(12) Patent Application Publication
(10) Pub. No.: US 2020/0354798 A1
(43) Pub. Date: Nov. 12, 2020

(54) METHODS FOR DETERMINING TUMOR MICROSATELLITE INSTABILITY

(71) Applicant: Illumina, Inc., San Diego, CA (US)

(72) Inventors: Li Liu, San Diego, CA (US); Shile Zhang, San Diego, CA (US); Mahdi Golkaram, San Diego, CA (US); Shannon Kaplan, San Diego, CA (US); Jie Ren, San Diego, CA (US)

(21) Appl. No.: 16/869,484

(22) Filed: May 7, 2020

Related U.S. Application Data
(60) Provisional application No. 62/845,415, filed on May 9, 2019.
(60) Provisional application No. 62/896,736, filed on Sep. 6, 2019.
(60) Provisional application No. 62/845,423, filed on May 9, 2019.
(60) Provisional application No. 62/900,929, filed on Sep. 16, 2019.

(51) Int. Cl. C12Q 1/6886

(52) U.S. Cl. CPC C12Q 1/6886; G01N 2800/60; C12Q 2600/158

(57) ABSTRACT

Presented herein are methods and compositions for determining MSI status and LS screening in a single test of colorectal cancer tissue. Also presented herein are methods for determining MSI status in a sample of cell-free DNA obtained from blood obtained from a cancer patient.

### CROSS-REFERENCE

This Application claims priority to U.S. Provisional Application Ser. No. 62/845,415, filed on May 9, 2019; U.S. Provisional Application Ser. No. 62/896,736, filed on Sep. 6, 2019; U.S. Provisional Application Ser. No. 62/845,423, filed on May 9, 2019; and U.S. Provisional Application Ser. No. 62/900,929, filed on Sep. 16, 2019, which applications are incorporated herein by reference in their entirety.

### FIELD OF THE INVENTION

The invention is applicable to the field of medical diagnostics and particularly relates to sequencing methods for identifying microsatellite instability and Lynch Syndrome in samples comprising tumor nucleic acids, including mixtures of tumor and normal cell-free DNA,

### BACKGROUND

Universal tumor screening for Lynch Syndrome (LS), which involves up to 6 sequential tests, is recommended by NCCN guidelines for all patients with colorectal cancer (CRC) at diagnosis. Microsatellite instability (MSI) testing is the first step in the genetic diagnosis for LS, which is most frequently linked to germline mutations in mismatch repair (MMR) genes or EPCAM. Additionally, MSI status has been approved by FDA to select patients for immunotherapy treatments.

Microsatellite instability (MSI) status has been approved by FDA to select for patients with metastatic tumors for cancer immunotherapy treatments. Additionally, MSI status is used in assessment of prognosis and treatment choices in certain cancer types, as well as the first step in the genetic diagnosis for Lynch syndrome. Circulating tumor DNA (ctDNA) is a noninvasive, real-time approach used for comprehensive genomic profiling of cancer. However, only a small fraction of cell-free DNA (cfDNA) fragments originate from tumor cells, requiring an ultra-sensitive method to detect MSI status from cfDNA

### FIGURES

FIG. 1 is a flow diagram for the LSFinder workflow from TSO500 tumor-only sequencing results.

FIG. 2 is a flow diagram for the analytical workflow to compute pathogenetic score for each MMR germline variant.

FIG. 3 is a decision tree for likelihood of carrying LS. pScore=pathogenetic score.

FIG. 4 is a chart showing TSO500 MSI scores for MSI-H and. MSS samples determined by .MSI-PCR. Dash line represents the MSI classification cutoff at 20.0.

FIG. 5 is a flow diagram showing the MSI analysis workflow of TSO500 cfDNA assay

FIG. 6 is a graph showing MSI score of MSI-H and MSS/normal samples (left) and zoomed in figure by removing MSI-H samples with MSI score >=2 (right). Dashed line is the established cutoff based on this cohort.

FIG. 7 chart showing MSI score of MSI-H samples across four different tumor types. Dashed line is the established cutoff based on this cohort.

FIG. 8 is a chart showing MSI score of four different titrated MSI-H cell lines. Dashed line is the established cutoff based on the cfDNA cohort.

### BRIEF SUMMARY

The present disclosure encompasses the discovery that a single tumor sequencing test using Illumina TruSigh™ Oncology 500 (TSO500) can detect both MSI status and LS screening.

The present disclosure also encompasses the discovery that sequencing of cell-free DNA obtained from blood of a patient having a tumor, using Illumina TruSight™ Oncology 500 (TSO500), test can accurately determine MSI status of the tumor.

The details of one or more embodiments are set forth in the claims and the description below. Other features, objects, and advantages will be apparent from the description, and from the claims.

### DETAILED DESCRIPTION

The present disclosure provides improved techniques for detecting and characterizing microsatellite instability using sequence data from samples of interest, and is related to the disclosure of International Application PCT/US2018/061067, filed on Nov. 14, 2018 and published as WO2019/099529A1, the entire contents of which are incorporated by reference herein.

As described in the incorporated materials of WO2019/099529A1, microsatellite instability may refer to the presence of nucleic acid replication errors in microsatellite repeat regions, which are short tandem repeat sequences (e.g., one to six base pairs in length) that are present throughout the genome. While microsatellite repeats may occur in untranslated regions of the genome, microsatellites may also be present in coding regions. During DNA replication, cells with microsatellite instability fail to repair DNA replication errors, which in turn may result in frame-shift mutations in the replicated daughter strand.

The presence of microsatellite instability may be associated with certain clinical conditions. For example, microsatellite instability is a hallmark of hereditary cancer syndrome, called Lynch Syndrome (LS), based on germline mutations of mismatch repair genes such as MLH1, PMS2, MSH2 and MSH6. Microsatellite instability status is typically assessed in clinical labs as an independent prognostic factor for favorable survival in cancer types such as colorectal and endometrial tumors. Further, certain treatment protocols or treatment options may be initiated to administer nivolumab or pembrolizumab for patients with solid tumors that have microsatellite instability high (MSL-H) designations or that are mismatch repair deficient (dMMR). Further, the treatment option may be to not administer pembrolizumab for patients with solid tumors that have microsatellite stable designations per a microsatellite instability score as determined herein. In another embodiment, the MSI typing (high, low, stable) may be used to determine whether a patient may benefit from adjuvant 5-fluorouracil (5-FU) chemotherapy. For colorectal cancer patients, adjuvant 5-fluorouracil (5-FU) chemotherapy may provide limited benefits in MSI-H patients. Therefore, an MSI-H designation may lead to cessation of or contraindication of adjuvant 5-fluorouracil (5-FU) chemotherapy. Such patients may instead be offered folinic acid, 5-FU and oxaliplatin. In another example, the MSI type of the patient may be used to determine if immunotherapy or chemotherapy is provided.

Accordingly, as provided herein, sequence data of samples of interest may be analyzed to determine a presence, absence, and/or degree of microsatellite instability in the sample of interest. Samples of interest with assessed microsatellite instability may be designated as MSI-H, microsatellite instability low (MSI-L), or microsatellite stable (MSS). The samples of interest may be tumor samples, and the microsatellite instability or stability designations may provide additional clinical information. As such, the present techniques may be used as part of or in conjunction with diagnosis, prognosis, and/or treatment protocols for cancer patients.

In certain embodiments, the present techniques permit assessment of samples of interest that do not have matched normal tissue samples. As provided herein, a reference sample dataset may be generated that is representative of a hypothetical matched normal sample for the sample of interest. The reference sample dataset may function as a universal matched normal sample. The reference sample dataset is generated from sequence data of the normal tissue of a plurality of individuals. When a tumor sample is tested, the appropriate reference sample dataset may be selected based on the tissue type, the sample origin, and other factors.

In certain embodiments, to generate a universal matched normal sample that may be applied to samples of interest independent of the ethnic background of the individual providing the sample, a reference sample dataset formed from samples of a multi-ethnic plurality of individuals (i.e., including individuals of a plurality of different ethnic backgrounds) may be assessed for microsatellite sites having relatively higher levels of variability between ethnic groups. Such sites may be eliminated or masked in the reference sample dataset, thus eliminating these highly variable sites from the analysis used to generate the overall microsatellite instability score representative of the sample of interest. In this manner, sites that are variable in normal samples due to variability between ethnic groups and not as a result of microsatellite instability do not introduce potentially erroneous results into the microsatellite instability score. Accordingly, the present techniques provide a more accurate microsatellite instability assessment for samples without a matched normal and independent of the ethnic background of the samples. In one example, the present techniques may be used to assess microsatellite instability for samples for which no ethnic background identification information is present. In another example, the reference sample dataset used as the hypothetical matched normal and that is generated with ethnically variable microsatellite regions filtered out of the dataset may be generally application to a wide variety of samples, thus eliminating additional processing steps or selection of an appropriate reference sample based on the ethnic background of the individual providing the sample of interest.

Presented herein are methods of determining MSI status and LS screening in a single test of colorectal cancer tissue. As exemplified by the non-limiting examples provided herein, in some embodiments, the method comprises sequencing a portion of the genome from the cancer sample, and quantifying mutations in the sample. In some embodiments, the method comprises sequencing a panel of 523 genes, covering at least 1.94 megabases (Mb). In some embodiments, the method comprises detecting microsatellite instability (MSI) and calculating a MSI score. In some embodiments, the method further comprises one or more of: detecting for BRAF p.V600E status by sequencing a portion of the BRAF gene, sequencing mismatch repair genes and detecting mutations therein, and sequencing EPCAM gene and detecting mutations therein.

In some embodiments, a LS diagnosis is based on based on a combination of 1) MSI-high (MSI-H) status, 2) without BRAF p.V600E mutations, and 3) at least 1 MMR number change.

Also presented herein are methods of determining MSI status in a sample of cell-free DNA obtained from blood obtained from a cancer patient. As exemplified by the non-limiting examples provided herein, in some embodiments, the method comprises sequencing a portion of the genome from the cancer sample, and quantifying mutations in the sample. In some embodiments, the method comprises sequencing a panel of 523 genes, covering at least 1.94 megabases (Mb). In some embodiments, the method comprises detecting microsatellite instability (MSI) and calculating a MSI score.

### EXAMPLE 1

### Microsatellite Instability Testing and Lynch Syndrome Screening For Colorectal Cancer Patients Through Tumor Sequencing

This example describes implementation of a method for determining MSI status and Lynch Syndrome status.

Background: Universal tumor screening for Lynch Syndrome (LS), which involves up to 6 sequential tests, is recommended by NCCN guidelines for all patients with colorectal cancer (CRC) at diagnosis. Microsatellite instability (MK) testing is the first step in the genetic diagnosis for LS, which is most frequently linked to germline mutations in mismatch repair (MMR) genes or EPCAM. Additionally, MSI status has been approved by FDA to select patients for immunotherapy treatments. Here we evaluate the performance of a single tumor sequencing test using alumina TruSight™ Oncology 500 (TSO500) for MSI status determination and LS screening.

Methods: A total of 233 CRC subjects were screened through a commercial MSI-PCR assay run on tumor-normal DNA. Tumor DNA from 63 selected subjects was sequenced with TSO500. The MSI score was calculated using 130 homopolymer microsatellite loci targeted by the TSO500 panel. Subsequently, BRAF p.V600E status and potential mutations in MMR genes or EPCAM were analyzed based on TSO500 results. For LS screening, a method with three filtering criteria was used: 1) MSI-high (MSI-H) status, 2) without BRAF p.V600E mutations, and 3) at least 1 MMR gene variant or EPCAM deletion inferred as germline small variant mutation or copy number change. Finally, matched normal samples were sequenced with TSO500 to confirm any germline mutations linked to LS.

Results: Using MSI-PCR, 45 of the 233 (19.3%) subjects were identified as MSI-H and 188 (80.7%) as microsatellite stable (MSS). TSO500 achieved an overall percent agreement (OPA) of 100.0% (95% CI: 94.3% 100.0%) with MSI-PCR for the 63 subjects analyzed by both methods. Eight subjects were identified as LS positive through tumor sequencing by TSO500. Matched normal sequencing confirmed all 8 positive cases of identified potential LS mutations as germline. Overall, TSO500 tumor sequencing achieved an OPA of 100.0% (95% Cl: 94.3%-100.0%) with matched normal sequencing.

Conclusions: Collectively, our results demonstrated that MSI status can be accurately determined with tumor sequencing. Moreover, LS screening by TSO500 can be used as a single upfront test to identify BRAF p.V600E status and potential pathogenic germline mutations linked to LS.

### EXAMPLE 2

### Microsatellite Instability Testing and Lynch Syndrome Screening For Colorectal Cancer Patients Through Tumor Sequencing

This example describes implementation of a method for determining MSI status and Lynch Syndrome status.

Background: Lynch Syndrome (LS) is most frequently linked to germline mutations in mismatch repair (MMR) genes MLH1, MSH2, MSH6, and PMS2 or EPCAM. Universal tumor screening for LS, which involves up to 6 sequential tests, is recommended by NCCN guidelines for all patients with colorectal cancer (CRC) at diagnosis¹. Microsatellite instability (MSI) testing is the first step in the genetic diagnosis for LS. Additionally, MSI status has been approved by FDA to select patients for immunotherapy treatments.

Illumina TruSight™ Oncology 500 (TSO500) is a target enrichment sequencing assay that enables comprehensive genomic profiling and measures tumor mutation burden (TMB) and microsatellite instability (MSI) in tissue samples through a tumor only workflow. TSO500 targets 523 genes, including all the genes that are associated with LS screening.

Here, we evaluate the performance of a single tumor sequencing test using TSO500 for MSI status determination and LS screening.

Methods: A total of 324 CRC subjects were screened using Promega MSI analysis system, which is a PCR-based assay run on tumor-normal DNA. Tumor DNA from 124 selected subjects were sequenced with TSO500. The MSI score was calculated using 130 homopolymer microsatellite loci targeted by the TSO500 panel. Subsequently, BRAT p.V600E status and potential mutations in MMR genes or EPCAM were analyzed based on TSO500 results.

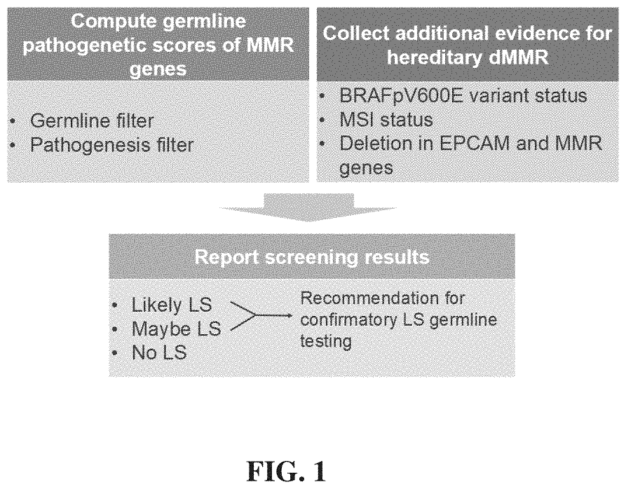

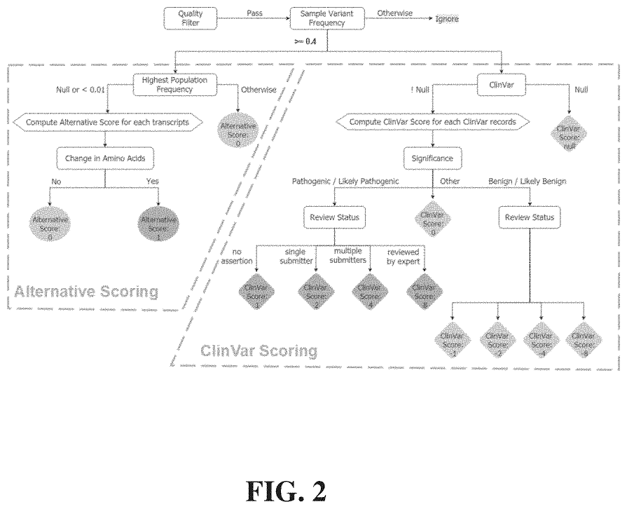

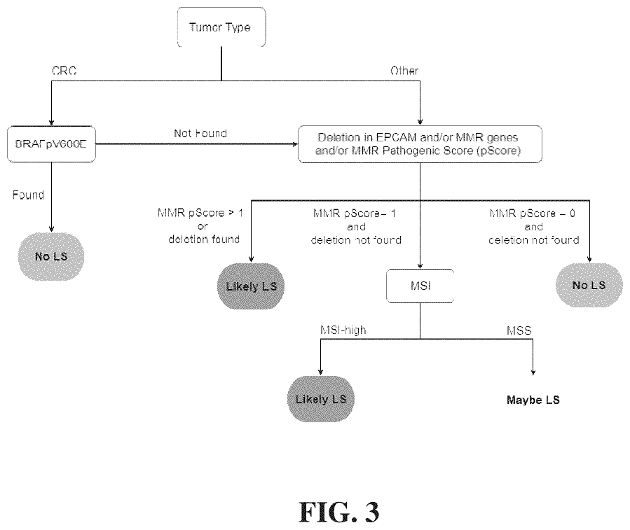

To determine LS status from TSO500 tumor-only sequencing, an in-house developed secondary analysis tool “LSFinder” was used (FIG. 1). Specifically, LS finder first determines if an MMR variant is germline using its variant allele frequency. For each MMR gerMline variant, it calculates a pathogenic score based on ClinVar scoring² and alternative scoring. The variant scores are then aggregated into a gene level pathogenic score (FIG. 2). The likelihood of a patient carrying L,S is determined based on MMR pathogenic scores, BRAF p.V600E status, MSI status, and potential deletions in MMR genes or EPCAM (FIG. 3). Finally, matched normal samples were sequenced with TSO500 to confirm any germline mutations linked to LS.

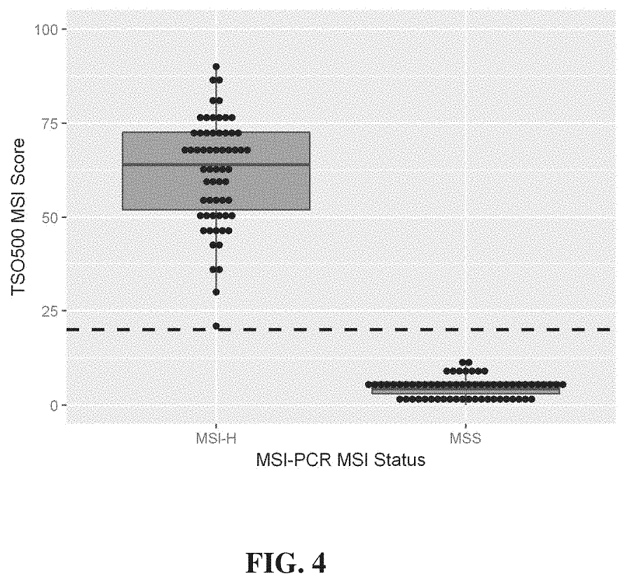

Results: Using MSI-PCR, 61 of the 324 (18.8%) subjects were identified as MSI high (MSI-H) and 263 (81.2%) as microsatellite stable (MSS). For the 124 selected subjects that were analyzed through TSO500, the assay achieved an overall percent agreement (OPA) of 100.0% (95% CI: 98.9%-100.0%) with MSI-PCR (FIG. 4 and Table 1).

TABLE 1
TSO500 MSI performance compared to MSI-PCR results.

MSI-PCR

| | | MSI-H | MSS |
|---|---|---|---|
| TSO500 | MSI-H | 61 | 0 |
| | MSS | 0 | 63 |

Upon running LSFinder on TSO500 tumor sequencing data, 5 subjects were identified as Likely LS, and 10 subjects as Maybe LS. All 15 potential positive cases were recommended for confirmatory germline testing (Table 2).

In the 109 subjects identified as No LS, BRAF p.V600E somatic mutation was found in 2 subjects, which could be falsely classified as LS positive through germline-only testing.

TABLE 2
LS screening results from TSO500 tumor-
only sequencing and recommendations

| | Number of CRC | |
|---|---|---|
| LS Status | Subjects | Recommendation |
| Likely LS | 5 | Confirmatory LS germline |
| Maybe LS | 10 | testing |
| No LS | 109 | No further testing |

Matched normal sequencing confirmed 13 of the 15 positive cases of identified potential LS mutations as germline. Two cases were determined as false positives as matched germline variants were not identified in normal sequencing. Overall, TSO500 tumor sequencing achieved an OPA of 98.4% (95% CI: 94.3%-99.8%) with matched normal sequencing (Table 3).

TABLE 3
Comparison of LS results from tumor only TSO500
sequencing with matched normal sequencing.

| Statistic | Value | 95% CI |
|---|---|---|
| PPA | 100% | 75.3%-100% |
| NPA | 98.2% | 93.6%-99.8% |
| PPV | 86.7% | 62.2%-96.3% |
| NPV | 100% | 96.7%-100% |
| OPA | 98.4% | 94.3%-99.8% |

PPA = positive percentage agreement, NPA = negative percentage agreement, PPV = positive predictive value, NPV = negative predictive value.

Conclusions: This example demonstrates MSI status can be accurately determined with TSO500 tumor-only sequencing. Genes associated with LS are covered by the TSO500 panel. TSO500 tumor sequencing with secondary analysis algorithms can be used as a single assay to identify BRAF p.V600E status and potential pathogenic germline variants linked to LS. In a study of 124 CRC subjects, TSO500 tumor sequencing achieved 100% OPA with MSI-PCR for MSI status determination, and 98.4% OPA and with matched normal sequencing for LS screening. Confirmatory germline testing was necessary for potential LS positive cases identified through TSO500.

### EXAMPLE 3

### Evaluation of Microsatellite Instability Testing Through Cell-Free DNA Sequencing

This example describes implementation of a method for determining MSI status of a tumor by sequencing of cell-free DNA.

Background: Microsatellite instability (MSI) status has been approved by FDA to select for patients with metastatic tumors for cancer immunotherapy treatments. Additionally, MSI status is used in assessment of prognosis and treatment choices in certain cancer types, as well as the first step in the genetic diagnosis for Lynch syndrome. Circulating tumor DNA (ctDNA) is a noninvasive, real-time approach used for comprehensive genomic profiling of cancer. However, only a small fraction of cell-free DNA (cfDNA) fragments originate from tumor cells, requiring an ultra-sensitive method to detect MSI status from cfDNA. Here we evaluate the performance of Illumina TruSight™ Oncology 500 panel for MSI testing through cfDNA sequencing.

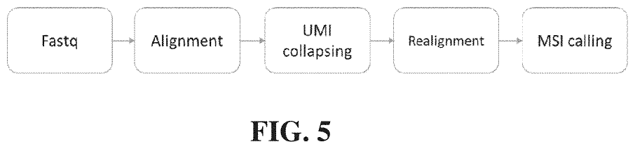

Methods: We developed a robust method to assess MSI status in cfDNA (FIG. 5). For each MSI locus, we assessed the repeat length distribution of the test subject and a cohort of normal samples. By comparing allele distributions using an information-theory based approach, we determined whether each MSI locus was unstable. The final MSI score was calculated as the number of unstable sites divided by the number of evaluable sites. To assess the analytical performance of our method, we titrated high (MSI-H) cell lines into MSI stable (MSS) background at a series of concentrations ranging from 0.31% to 5.0%, representing low tumor fractions in cfDNA samples. Additionally, we processed 136 clinical samples with matched FFPE tumor and cfDNA to examine the concordance of MSI testing between FFPE and cfDNA.

Results: For titrated MSI-H samples with low tumor fraction, we achieved 100% sensitivity at 0.625% MSI-H content titration into MSS background. Moreover, we achieved 100% overall percent agreement (93/94) for MSI status between matched FFPE and cfDNA samples with a wide range of tumor content.

Conclusions: Our evaluation indicates that we can accurately determine MSI status in cIDNA samples with a wide range of tumor content.

The following paragraphs give further detail to the paragraphs above.

Using the Trusight™ Oncology 500 (TSO 500) assay targeting 523 genes, molecular profiling was performed with unique molecular identifiers (UMIs), sequenced on Illumina platforms, and analyzed using an internal pipeline. Our algorithm to determine microsatellite instability status from a cfDNA sample is summarized as follows:
Utilizing UMIs to collapse duplicate read families into a single consensus sequence to achieve lower error rates
Collapsed sequences supported by reads from both the forward and reverse strand were used for allele distribution calculation of each MSI site
Unstable microsatellite sites were detected by assessing the shift in the length of a microsatellite site for a tumor sample against 48 normal baseline samples using an information-theory based approach
Final MSI score was calculate using the sum of distance derived from unstable microsatellite sites compared between tumor and normal baseline samples

To establish the algorithm and MSI score cutoff, we assessed 136 subjects through cfDNA sequencing. Nine samples (5 colorectal, 2 endometrial, 1 prostate and 1 lung) with tumor content >1% were identified as MSI-high (MSI-H) through matched FFPE sequencing or vendor information. To assess reproducibility and robustness of our assay and algorithm, we have sequenced selective samples multiple times (Table 4).

TABLE 4
cfDNA sample summary table

| MSI status | unique sample | data points |
|---|---|---|
| normal or MSS | 127 | 275 |
| MSI-H | 9 | 19 |

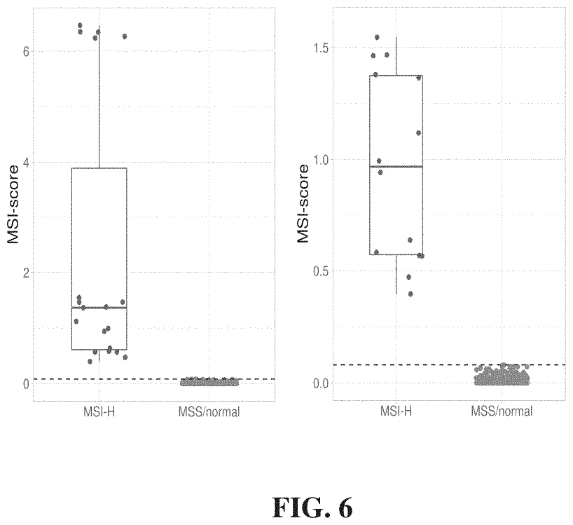

Utilizing >1000 sites with the 523 gene panel, we calculated the MSI score based on cfDNA sequencing and achieved total separation between MSI-H and MSS/normal samples (FIG. 6). Subsequently, we set MSI score cutoff at 0.08 for further analysis.

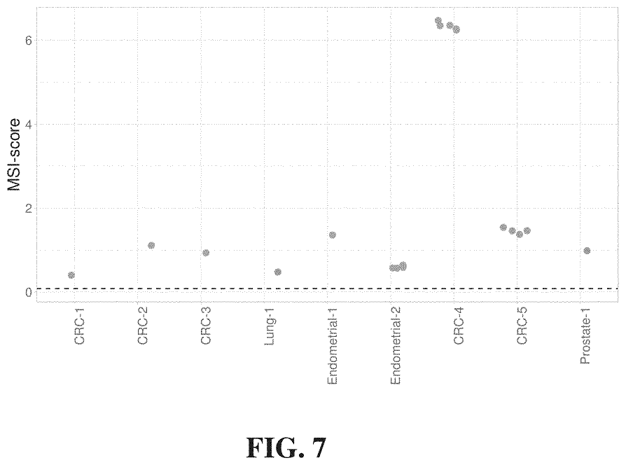

Although our MSI-H cfDNA cohort is small (n=9), we have successfully detected MSI-1-1 samples across four different tumor types including CRC, Endometrial, Prostate and Lung. Furthermore, repeated sequencing of the same samples (Sample IDs Endometrial-2, CRC-4 and CRC-5) demonstrated high reproducibility based on our assay and algorithm (FIG. 7).

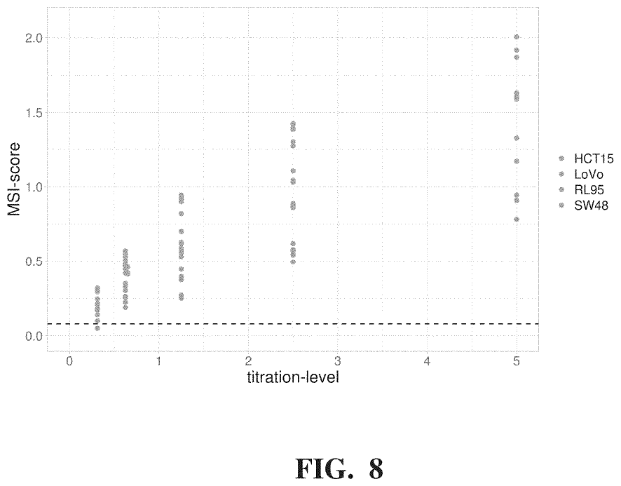

Here we assessed the analytical performance of our method, by titrating four different MSI-H cell lines into MSS background at a series of concentrations ranging from 0.31% to 5.0%. For three of the four cell lines, we achieved 100% sensitivity at all titration levels. Additionally, we achieved 100% sensitivity at 0.625% for all cell lines on all technical replicates (FIG. 8 and Table 5).

TABLE 5
Analytical performance based on four
different titrated MSI-H cell lines.

| cell line | | data | |
|---|---|---|---|
| titration level | unique cell line | point | sensitivity |
| 0.31% | 4 | 2 | 91.7% |
| 0.63% | 4 | 8 | 100% |
| 1.25% | 4 | 1 | 100% |
| 2.50% | 4 | 1 | 100% |
| 5.00% | 4 | 2 | 100% |

Collectively, our evaluation indicates that we can accurately determine MSI status in cfDNA samples with a wide range of tumor content.

A number of embodiments have been described. Nevertheless, it will be understood that various modifications may be made. Accordingly, other embodiments are within the scope of the following claims.

What is claimed is:

1. A method of simultaneously determining clinically relevant MSI status and detecting Lynch Syndrome in a colorectal cancer sample comprising:
a) detecting a marker of MSI status in a cancer sample from a subject;
b) detecting for BRAF p.V600E status and potential mutations in mismatch repair (MMR) genes or EPCAM; and
c) identifying the subject as having Lynch Syndrome based on the results of a) and b).

2. The method of claim 1, wherein the step of detecting comprises sequencing a portion of the genome from the cancer sample, and determining MSI status based on the sequencing.

3. The method of claim 2, wherein the portion of the genome comprises at least 40, 50, 60, 70, 80, 90, 100, 110, 120, 130, 140, 150 or more homopolymer microsatellite loci.

4. The method of claim 3, wherein the portion of the genome comprises at least 130 homopolymer microsatellite loci.

5. The method of claim 1, wherein detecting for BRAE p.V600E status comprises sequencing a portion of the BRAF gene.

6. The method of claim 5, further comprising sequencing mismatch repair genes and detecting mutations therein.

7. The method of claim 5, further comprising sequencing EPCAM gene and detecting mutations therein.

8. The method of claim 2, wherein the portion of the genome covers at least 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9 megabases (Mb) of the genome.

9. The method of claim 8, wherein the portion of the genome covers at least 1.9 megabases (Mb).

10. The method of claim 9, wherein the portion of the genome covers at least 1.94 megabases (Mb).

11. The method of any of claims 1-10, further comprising classifying t colorectal cancer sample as Lynch Syndrome positive if the following are true:
a MSI-high (MSI-H) status,
b. without BRAF p.V600E mutations, and
c. at least 1 MMR gene variant or EPCAM deletion inferred as germline small variant mutation or copy number change.

12. A method of determining clinically relevant MSI status from cell-free DNA sample comprising: detecting a marker of MSI status in a cell-free DNA sample obtained from a subject with cancer.

13. The method of claim 12, wherein the step of detecting comprises sequencing a portion of the genome from the cancer sample, and determining MSI status based on the sequencing.

14. The method of claim 13, wherein the portion of the genome comprises at least 40. 50, 60, 70, 80, 90, 100, 110, 120, 130, 140, 150 or more homopolymer microsatellite loci.

15. The method of claim 14, wherein the portion of the genome comprises least 130 homopolymer microsatellite loci.

16. The method of claim 13, wherein the portion of the genome covers at least 0.1, 0.2, 0.3, 0.4, 0.5, 0.6, 0.7, 0.8, 0.9, 1.0, 1.1, 1.2, 1.3, 1.4, 1.5. 1.6, 1.7, 1.8, 1.9 megabases (Mb) of the genome.

17. The method of claim 16, wherein the portion of the genome covers at least 1.9 megabases (Mb).

18. The method of claim 17, wherein the portion of the genome covers at least 1.94 megabases (Mb).

19. The method of any of claims 12-18, wherein determining MSI status comprises, for each locus of a plurality of MSI loci, assessing the repeat length distribution and comparing to repeat length distribution of a cohort of normal samples.

20. The method of any of claims 12-19, comprising
comparing allele distributions using an information-theory based approach, thereby determining whether each MSI locus was unstable; and
calculating a parameter between the number of unstable sites and the number of evaluable sites.
